(19) United States
(12) Patent Application Publication
(10) Pub. No.: US 2026/0314589 A1
(43) Pub. Date: Oct. 15, 2026

(54) Kicking Waterfowl Decoy

(71) Applicant: Good Sportsman Marketing, LLC, Irving, TX (US)

(72) Inventors: Fred L. Zink, JR., Port Clinton, OH (US); Damon L. Coalson, Dallas, TX (US)

(21) Appl. No.: 19/640,346

(22) Filed: Apr. 6, 2026

Related U.S. Application Data
(60) Provisional application No. 63/786,168, filed on Apr. 9, 2025.

(51) Int. Cl. A01M 31/06

(52) U.S. Cl. CPC A01M 31/06

(57) ABSTRACT

An assembly for use on a water surface includes a decoy for a waterfowl. The decoy is buoyant on the water surface and has articulating feet connected at hinged connections to the decoy. An actuator is disposed in a sealed chamber of the decoy and includes a motor and a converter. The motor is being operable to produce a drive, and the converter is coupled between the motor and the articulating feet and is configured to convert the drive of the motor into reciprocating motion of the articulating feet at the hinged connections. A power supply is disposed in the sealed chamber and is configured to provide power. Control circuitry disposed in the sealed chamber is configured to operate the motor with the power from the power supply.

### CROSS-REFERENCE TO RELATED APPLICATIONS

This application claims the benefit of U.S. Provisional Appl. No. 63/786,168 filed Apr. 9, 2025, which is incorporated herein by reference in its entirety.

### BACKGROUND OF THE DISCLOSURE

Decoys for waterfowl can be used to create the illusion of a safe, attractive gathering spot where the waterfowl can land, feed, or rest. For example, ducks are social birds and tend to flock toward other ducks when migrating or looking for safe places to land. When ducks see what looks like a group already on the water, the ducks are more likely to come in range of the hunters, believing it is a safe and active spot.

The use of decoys for waterfowl, such as ducks, is thousands of years old and is one of the oldest hunting tools in North America. Earliest decoys dating back over 2,000 years were made by Native American tribes (like the Paiute) and have been found in caves around present-day Nevada. These decoys used bulrushes and feathers. In the 1800s and early 1900s, hunters made hand-carved, hand-painted wooden decoys, and decoy carving became an American folk art.

Modern decoys today are often made of plastic, foam, or lightweight materials, with detailed paint jobs and sometimes built-in movement features. Traditionally, decoys are buoyant devices that merely float on the water surface and may be anchored in place using anchors. Hunters strategically place multiple decoys (called a spread) on the water in realistic patterns to mimic the behavior of live ducks. Hunters use patterns, numbers, and positioning in the spread based on duck behavior, weather, and location. The decoys can be stationary or motion-based (like spinning wing decoys) to add movement and make the setup more convincing.

Hunters may use species-specific decoys (mallards, pintails, teal) to match local flocks. Early in the season, hunters may use smaller spreads but may use much larger spreads late in the season when ducks are flocking heavily. Different spread patterns can be used. In J-Hook or Fishhook patterns, for example, ducks tend to land at the open end of the hook. In U-Shape or Horseshoe patterns, ducks naturally land in the opening. Ducks prefer to land into the wind so hunters will often leave an open pocket upwind, steering the ducks toward that area. Additionally, the decoys are usually spaced with enough room between them to look realistic.

Ducks are social and cautious. If the ducks see decoys that sit stiff in flat, still water, the ducks get suspicious. But if there is natural-looking movement, splashes, and ripples, the ducks are much more likely to commit and land. To make the decoys more lifelike to attract waterfowl, various techniques have been used.

In one of the oldest and simplest techniques, a jerk string is attached to a few decoys and anchored with a weight. The hunter sits in the blind holding the end of the line. When hunter tugs the string, the decoys bob, ripple the water, and mimic the subtle movement of feeding ducks.

To make the decoys more lifelike to attract waterfowl, various motorized mechanisms have been added to the decoys to mimic flapping wings, feeding movements, and the like. For example, hunters may use motion decoys that include spinning wings or swimming decoys to add natural movement, especially on calm days when the water is still. Some brief examples are disclosed in US5377439, US6088944, US6412209, US2004/0010957, US2006/0207158, US2014/0259860, US2017/0049095, USUS11083189, and US2023/0380411.

For example, in one mechanized technique, a pulsator or bubbling decoy is battery-powered and has a small pump inside. The decoy sits low in the water and periodically pulse water jets underneath, creating ripples and movement.

A feeder butt decoy is shaped like an upside-down duck, showing just the tail and rear end sticking up—exactly like a duck tipping forward to feed. Hunters often include a few of these to visually signal to other ducks that the water is a safe, food-rich spot.

A swimmer decoy actually floats in small circles or moves forward slowly via a motor or cord. This imitates the subtle back-and-forth or slow drifting movement ducks make while nibbling around the water’s surface.

To make ripples, separate devices (e.g., floating battery-powered pucks) can be used to create gentle ripples and movement across the whole spread. Even stationary decoys look more lifelike when the water surface is not perfectly still. Finally, a wind-driven decoy can have small paddles underneath that spin when the wind blows, causing the decoy to bob, rock, or splash lightly.

Although the above techniques may be effective, hunters are always striving to making decoy look as “alive” and natural as possible.

The subject matter of the present disclosure is directed to overcoming, or at least reducing the effects of, one or more of the problems set forth above.

### SUMMARY OF THE DISCLOSURE

An assembly disclosed herein is used to mimic a waterfowl on a water surface. The assembly comprises a decoy, at least one articulating foot, an actuator, and control circuitry. The decoy has a sealed chamber and is buoyant with at least a portion of the decoy above the water surface. The at least one articulating foot is connected to the decoy, and the actuator is disposed in the sealed chamber. The actuator comprises a motor and a converter. The motor is operable to produce a drive, and the converter is coupled between the motor and the at least one articulating foot. The converter is configured to convert the drive of the motor into reciprocating motion of the at least one articulating foot. For example, the reciprocating motion of the at least one articulating foot can move at least portion of the at least one articulating foot above and below the water surface.

The control circuitry in electrical communication with the actuator is configured to operate the motor. The sealed chamber can house at least a portion of the control circuitry, and the decoy can comprise a buoyant chamber being configured to provide buoyancy to the decoy. The control circuitry can comprise a power supply disposed in the sealed chamber.

The assembly can further comprise an anchor useable below the water surface. The anchor can have a tether connected to the decoy at the water surface. The anchor can house at least a portion of the control circuitry so the tether can have conductors connected to the actuator. The anchor can house a power supply so the tether can have conductors conducting power from the power supply to the actuator.

The converter can comprise a gear reducer reducing rotation of the drive from the motor. The motor can produce rotation for the drive in one direction, and the converter can convert the rotation in the one direction to the reciprocating motion. Alternatively, the motor can produce rotation for the drive in first and second directions opposing one another. The converter can convert the rotation in the first direction into an advance of the reciprocating motion and can convert the rotation in the second direction into a return of the reciprocating motion.

In one arrangement, the assembly can comprise two of the at least one articulating foot. Each of the articulating feet can be connected at a rotatable connection to the decoy. In this arrangement, the motor is operable to produce the drive for both of the articulating feet, and the converter coupled between the motor and the articulating feet is configured to convert the drive of the motor into the reciprocating motion of both of the articulating feet at the rotatable connections.

In one configuration, the converter can comprise crank wheels turned by the drive of the motor. Each of the crank wheels is connected by a first hinge to a linkage arm, and each linkage arm is connected by a second hinge to a lever. Each lever is connected to an axle of a respective one of the rotatable connections for a respective one of the articulating feet.

In other configurations, the converter can comprise: (i) a crank and a slider, the crank being rotatable in response to the drive and being connected to the slider via a connecting rod, the slider being connected to the rotatable connection of the at least one articulating foot; (ii) a scotch yoke having a wheel and a reciprocating shaft, the wheel being rotatable in response to the drive and having a pin engaged in a slot of the reciprocating shaft, the reciprocating shaft being connected the rotatable connection of the at least one articulating foot; (iii) a cam and a follower, the cam being rotatable in response to the drive and moving the follower back and forth, the follower being connected to the rotatable connection of the at least one articulating foot; (iv) a rack and a pinion with an oscillating link; (v) a rotating wheel being rotatable in response to the drive, the rotating wheel driving a Geneva wheel in discrete steps that drive a linear mechanism connected to the rotatable connection of the at least one articulating foot; and (vi) a lead screw rotated by the drive, the lead screw being configured to move a slider connected to the rotatable connection of the at least one articulating foot, limit switches being configured to reverse rotation of the motor at end of travel of the slider.

The control circuitry can comprise at least one of: a timer configured to intermittently operate the motor; and a control configured to control a speed of the motor.

The assembly can further comprise another motor disposed in the sealed chamber and being operable to produce oscillations of the decoy. The control circuitry in electrical communication with the other motor can be configured to operate the other motor.

Another assembly disclosed herein is for use on a water surface. The assembly comprises a decoy, articulating feet, an actuator, a power supply, and control circuitry. The decoy has a sealed chamber and is buoyant with at least a portion of the decoy above the water surface. The articulating feet are connected at rotatable connections to the decoy, and the actuator is disposed in the sealed chamber.

The actuator comprises a motor and a converter. The motor is operable to produce a drive. The converter is coupled between the motor and the articulating feet and is configured to convert the drive of the motor into reciprocating motion of the articulating feet at the rotatable connections. The power supply is disposed in the sealed chamber and is configured to provide power. The control circuitry is disposed in the sealed chamber and is in electrical communication with the actuator and the power supply. The control circuitry is configured to operate the motor with the power from the power supply.

Yet another assembly disclosed herein is for use on a water surface. The assembly comprises a decoy, articulating feet, an actuator, an anchor, and control circuitry. The decoy has a sealed chamber and is buoyant with at least a portion of the decoy above the water surface. The articulating feet are connected at rotatable connections to the decoy, and the actuator is disposed in the sealed chamber. The actuator comprises a motor and a converter. The motor is operable to produce a drive, and the converter is coupled between the motor and the articulating feet. The converter is configured to convert the drive of the motor into reciprocating motion of the articulating feet at the rotatable connections.

The anchor is useable below the water surface and is connected to the decoy by a tether. The anchor has a power supply configured to provide power, and the tether conducts the power from the power supply to the actuator. The control circuitry is in electrical communication with the actuator and the power supply. The control circuitry is configured to operate the motor with the power from the power supply.

The foregoing summary is not intended to summarize each potential embodiment or every aspect of the present disclosure.

### BRIEF DESCRIPTION OF DRAWINGS

FIG. 1A illustrates a waterfowl decoy assembly according to the present disclosure.

FIG. 1B illustrates another waterfowl decoy assembly according to the present disclosure.

FIG. 2A illustrates side view of a waterfowl decoy of the assembly in FIG. 1A.

FIG. 2B illustrates a back view of the waterfowl decoy.

FIG. 2C illustrates a bottom view of the waterfowl decoy.

FIG. 3A illustrates an elevational view of the sealed housing for the disclosed waterfowl decoy.

FIG. 3B illustrates a bottom view of the sealed housing for the disclosed waterfowl decoy.

FIG. 4 illustrates a cross-sectional view of the waterfowl decoy along section I-I in FIG. 2A.

FIG. 5A illustrates a bottom view of the sealed housing for the disclosed waterfowl decoy.

FIG. 5B illustrates a cross-sectional view of the sealed housing along section II-II in FIG. 5A.

FIG. 6A illustrates a bottom view of the sealed housing for the disclosed waterfowl decoy.

FIG. 6B illustrates a cross-sectional view of the sealed housing along section III-III in FIG. 6A.

FIG. 7A illustrates a bottom view of the sealed housing for the disclosed waterfowl decoy.

FIG. 7B illustrates a cross-sectional view of the sealed housing along section IV-IV in FIG. 7A.

FIGS. 8A-8D diagram various mechanisms for the converter of the disclosed decoy to convert rotation of the motor into reciprocation of the feet.

FIG. 9 schematically illustrates control circuitry for the disclosed waterfowl decoy assembly.

### DETAILED DESCRIPTION OF THE DISCLOSURE

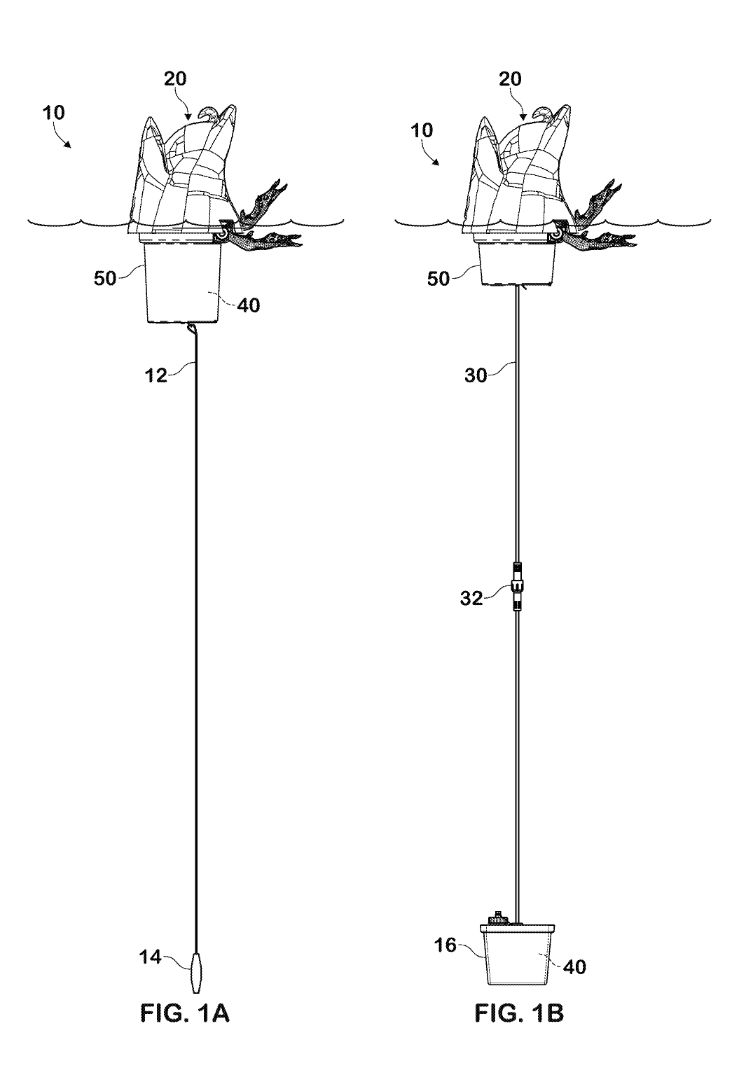

FIG. 1A illustrates a waterfowl decoy assembly 10 according to the present disclosure, and FIG. 1B illustrates another waterfowl decoy assembly 10 according to the present disclosure. The waterfowl decoy assembly 10 mimics a waterfowl on a water surface. The waterfowl decoy assembly 10 includes a decoy 20 that is buoyant on the water surface. At least a portion of the decoy 20 extends above the water surface. The decoy 20 has articulating feet 24a-b and an actuator 50.

The decoy 20 is designed to mimic a waterfowl, floating on the water surface and tipped down below the water surface to feed below the surface. As can be seen, the decoy 20 is made to resemble a waterfowl’s tail end protruding from the water surface to mimic the waterfowl dipping its head under the water surface to feed. While feeding, ducks tend to paddle and splash the water for balance. The articulating feet 24a-b are articulated by the actuator 50 to kick in a reciprocating motion, which produces a wake in the water surface.

For example, the decoy 20 emulates a duck feeding just below the surface of the water. The decoy 20 has a duck’s tail for decoration, and the actuator 50 drives articulating feet 24a-b in a kicking motion. The decoy 20 is designed to emulate the look and motion of a real duck. When the wind is slight, the decoy 20 moves with lifelike motion and creates waves that bounce off adjacent decoys creating the look of live ducks.

For the waterfowl decoy assembly 10 of FIG. 1A, the waterfowl decoy 20 floating at the water surface includes a power supply 40, which powers the actuator 50. An anchor line 12 can connect to the decoy 20 and can have an anchor or weight 14 for placement. In FIG. 1B, the waterfowl decoy assembly 10 includes an anchor 16 having the power supply 40. A tether 30 having conductors connects the power supply 40 in the anchor 16 to the actuator 50 on the decoy 20. Connectors 32 may be used on the tether 30.

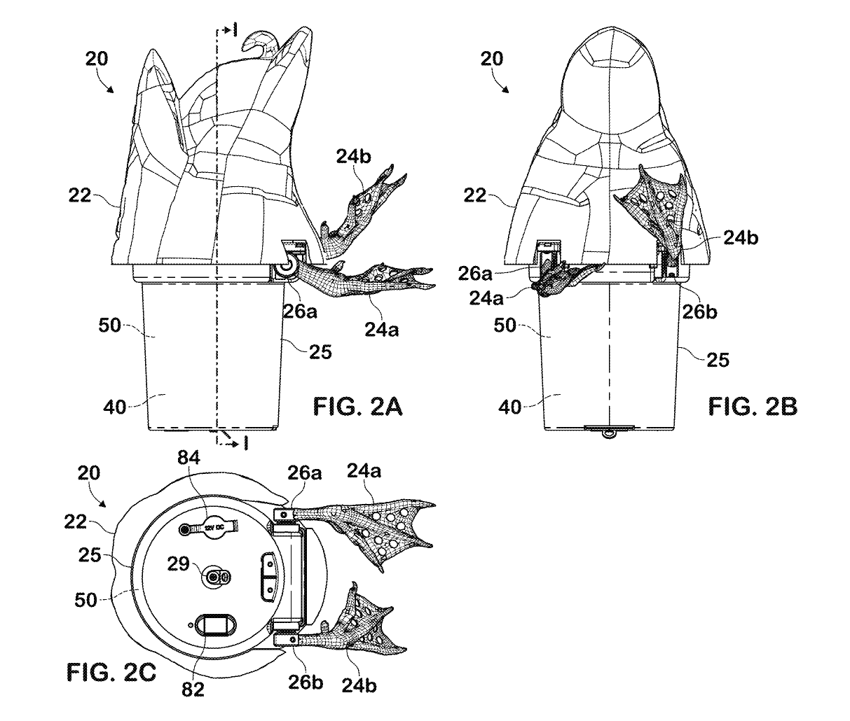

Turning to more details of the decoy 20, FIG. 2A illustrates side view of the waterfowl decoy 20 of the assembly in FIG. 1A. FIG. 2B illustrates a back view of the waterfowl decoy 20, and FIG. 2C illustrates a bottom view of the waterfowl decoy 20.

The waterfowl decoy 20 includes a decoy body 22, which mimics the tail end of the waterfowl and extends above the water surface. The waterfowl decoy 20 also includes a sealed housing 25 in which the actuator 50 and power supply 40 are disposed. The articulating feet 24a-b are connected at rotatable or hinged connections 26a-b to the decoy 20. Again, being articulated by the actuator 50, the articulating feet 24a-b kick in a reciprocating motion.

As shown in the bottom view of FIG. 2C, the sealed housing 25 of the decoy 20 can include a fixture 29 for connection to an anchor line. Additionally, the sealed housing 25 can include a power button 102 and a charging port 104, which can be sealed to prevent water leaking into the sealed housing 25.

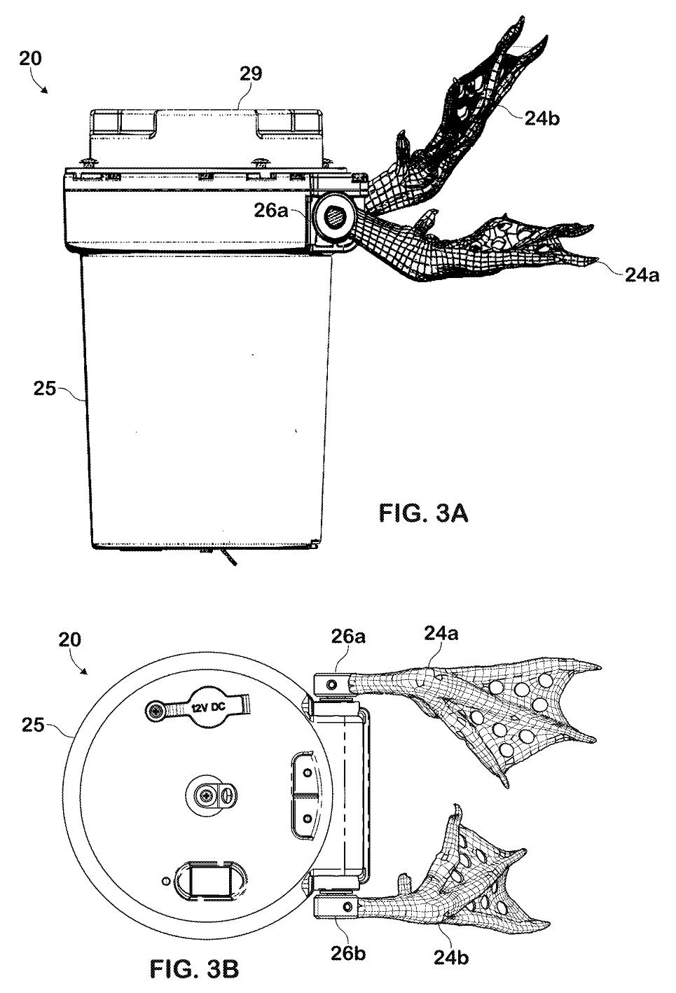

FIG. 3A illustrates an elevational view of the sealed housing 25 for the disclosed waterfowl decoy 20, and FIG. 3B illustrates a bottom view of the sealed housing 25 for the disclosed waterfowl decoy 20. A top 28 seals to the sealed housing 25 so that the sealed housing 25 is waterproof. The sealed housing 25 can hold all of the internal components inside, and the decoy body (22) can affix to the top 28 of the sealed housing 25.

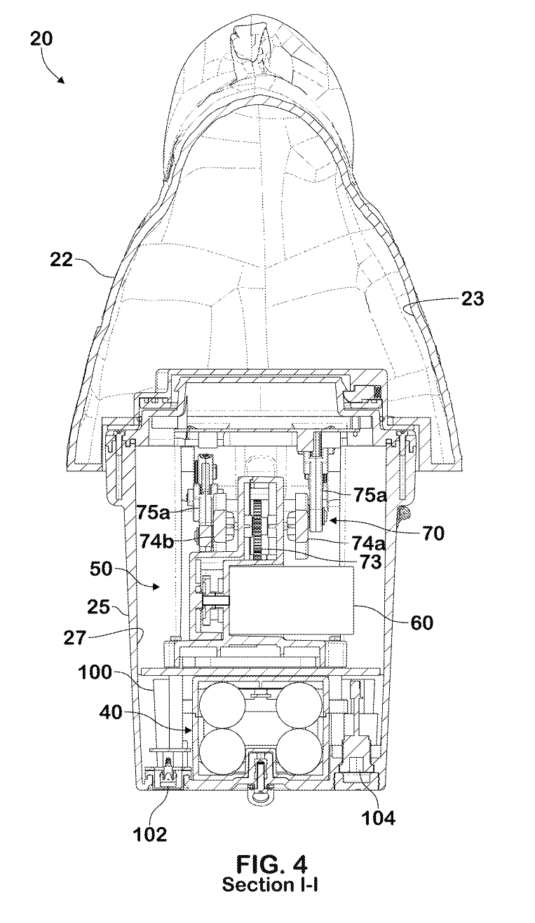

FIG. 4 illustrates a cross-sectional view of the waterfowl decoy 20 along section I-I in FIG. 2A. As shown, the sealed housing 25 of the decoy 20 includes a sealed chamber 27, which houses the power supply 40 and the actuator 50. The power supply 40 can include one or more rechargeable batteries, which can be charged by the charging port 104. The power button 102 can be part of control circuitry 100 also housed in the sealed chamber 27. As further shown, the body 22 of the decoy 20 can define a buoyant chamber 23, which may provide additional buoyancy to the decoy 20.

The actuator 50 includes a motor 60 and a converter 70. Preferably, one motor 60 is used to reciprocate both feet 24a-b, although it would be possible for each foot 24a-b to be reciprocated by a separate motor 60. In this example, one motor 60 is used. As noted in other examples, the power supply 40 can be housed in a separate anchor (16) and connected by conductors in a tether to the actuator 50. The control circuitry 100 can also be housed in the separate anchor (16) along with the power supply 40.

In this example, the power supply 40 and the control circuitry 100 are housed in the sealed housing 25. The control circuitry 100 is in electrical communication with the power supply 40 and is configured to operate the motor 60. When operated, the motor 60 produces a drive, and the converter 70, which is coupled between the motor 60 and the articulating feet (24a-b), is configured to convert the drive of the motor 60 into reciprocating motion of the articulating feet 24a-b at the rotatable connections 26a-b.

The motor 60 can be an electric motor, and a reducer (e.g., set of gears) can be used to reduce the rotation of the drive from the electric motor 60. The power supply 40 can use lithium-Ion batteries to power the electric motor 60. The control circuitry 100 can include hardware (or a processor with software) to control operation. The control circuitry 100 can include communication features, such as wireless communication, so a hunter can operate the decoy 20 with remote control. The control circuitry 100 can include one or more timers configured to control the motor 60, such as intermittently operating the motor 60. The control circuitry 100 can also be configured to control the speed of the motor 60, changing the speed from faster to slower.

In one configuration, the motor 60 rotates with the drive in one direction, and the converter 70 converts the drive in the one direction into the reciprocating motion, making the articulating feet 24a-b kick up and down about the rotatable connections 26a-b. In another configuration, the motor 60 rotates with the drive in opposing directions (i.e., clockwise and counterclockwise). The converter 70 converts the drive in the clockwise direction into an advance of the reciprocating motion and converts the drive in the counterclockwise direction into a return of the reciprocating motion so that the articulating feet 24a-b kick up and down about the rotatable connections 26a-b. For simplicity, the motor 60 rotates with the drive in one direction, and the converter 70 converts the drive in the one direction into the reciprocating motion.

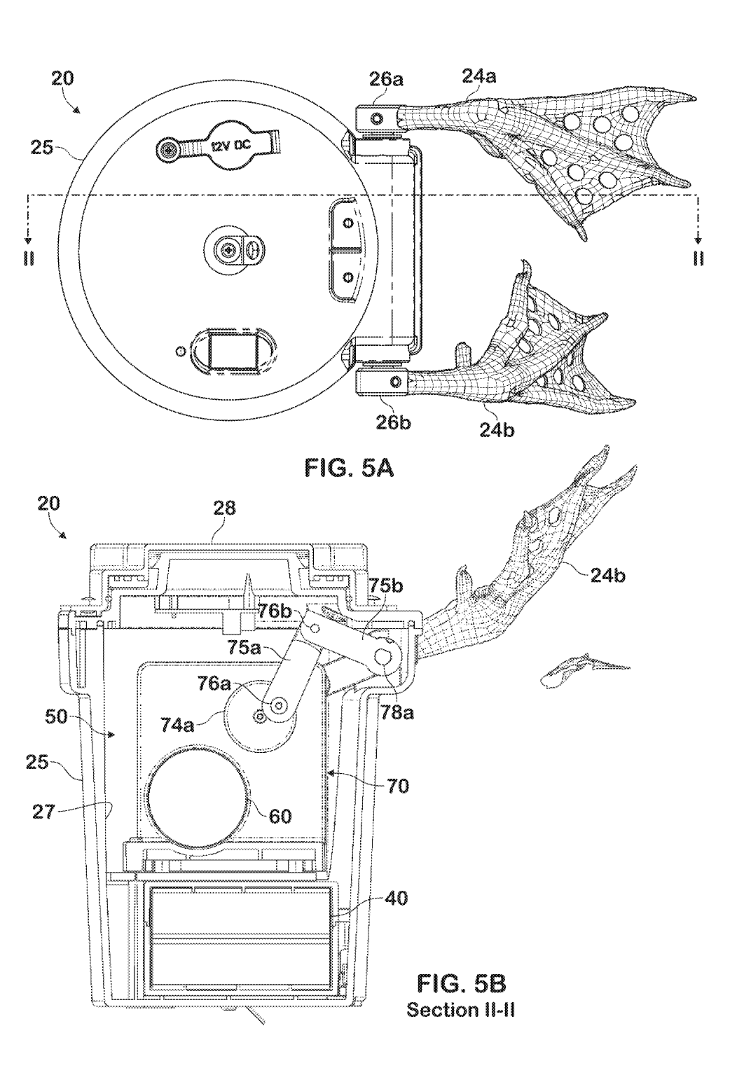

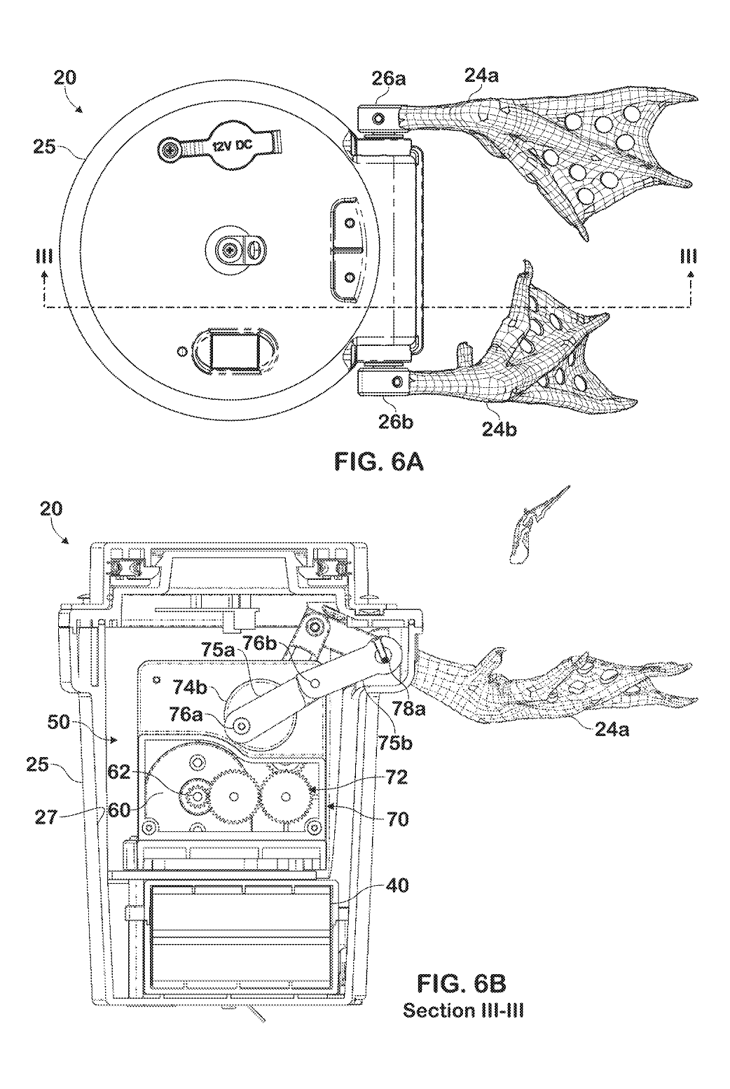

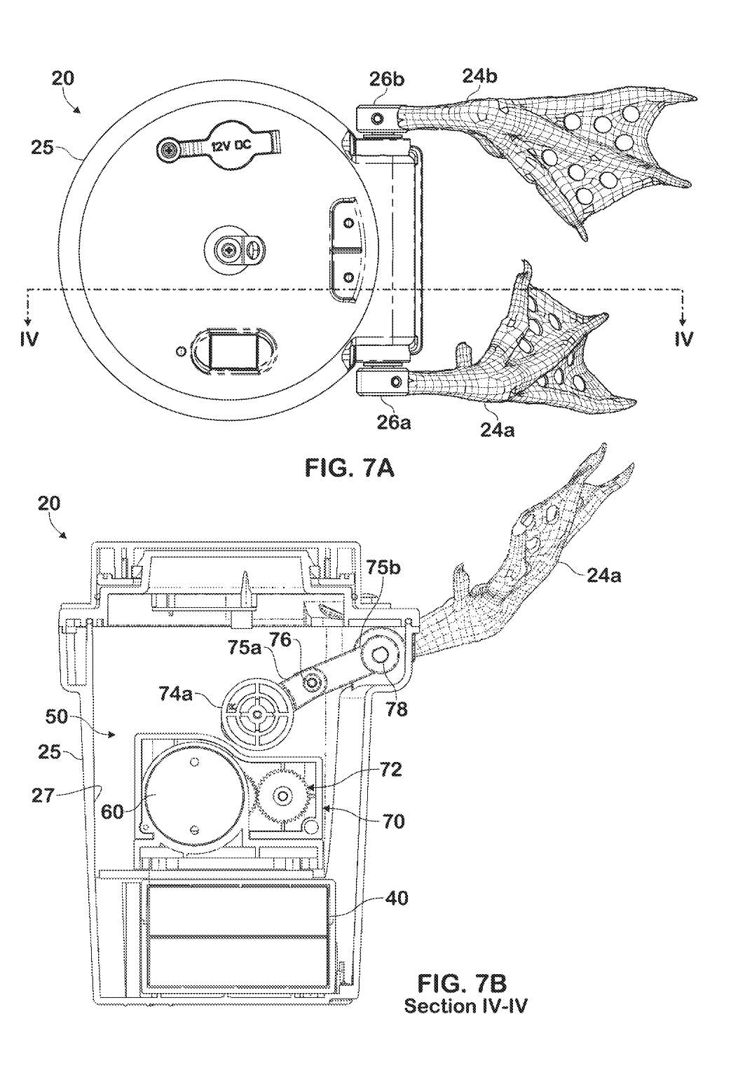

Various mechanisms can be used for the converter 70 to convert rotation in one direction from the motor 60 into reciprocation of the feet 24a-b. As shown in FIGS. 5A through 7B, one configuration of the converter 70 uses a crank and lever mechanism. In particular, FIGS. 5A, 6A, and 7A illustrate bottom views of the sealed housing 25 for the disclosed waterfowl decoy 20. Meanwhile, FIG. 5B illustrates a cross-sectional view of the sealed housing 25 along section II-II in FIG. 5A, and FIG. 6B illustrates a cross-sectional view of the sealed housing 25 along section III-III in FIG. 6A. Finally, FIG. 7B illustrates a cross-sectional view of the sealed housing 25 along section IV-IV in FIG. 7A.

Overall, the converter 70 includes a series of gears 72, a pair of crank wheels 74a-b, linkage arms 75a, levers 75b, and a pair of axles 78 connected between the motor 60 and the two articulating feet 24a-b for kicking the water surface. As shown in FIG. 6B, the shaft 62 of the motor 60 connects to a series of gears 72. In turn, these gears 72 connect to a central gear 73 as shown in FIG. 4, which connects with and rotates the pair of crank wheels 74a-b—one for each foot 24a-b.

FIG. 5B shows the first crank wheel 74a for the first foot 24a. A linkage arm 75a connects by a hinge 76a to the first crank wheel 74a and connects by another hinge 76b to the lever 75b. The lever 75b is connected to a first axle 78a to which a first rotatable or hinged connection 26a for the first foot 24a connects. The axle 78a can have a flat side, and the first rotatable or hinged connection 26a can affix with an Allen nut or the like against the flat side of the axle 78a. Turning of the first crank wheel 74a cranks the linkage arm 75a, which reciprocates the lever 75b to turn the axle 78a back and forth so that the connected foot 24a kicks up and down.

The arrangement for the other foot 24b is similar. For example, FIG. 6B shows a second crank wheel 74b for the second foot 24b. A linkage arm 75a connects by a hinge 76a to the second crank wheel 74b and connects by another hinge 76b to the lever 75b. The lever 75b is connected to a second axle 78b to which a second rotatable or hinged connection 26b for the second foot 24b connects. Again, the second axle 78b can have a flat side, and the second rotatable or hinged connection 26b can affix with an Allen nut or the like against the flat side of the axle 78b. Turning of the second crank wheel 74b cranks the linkage arm 75a, which reciprocates the lever 75b to turn the axle 78b back and forth so that the connected foot 24b kicks up and down.

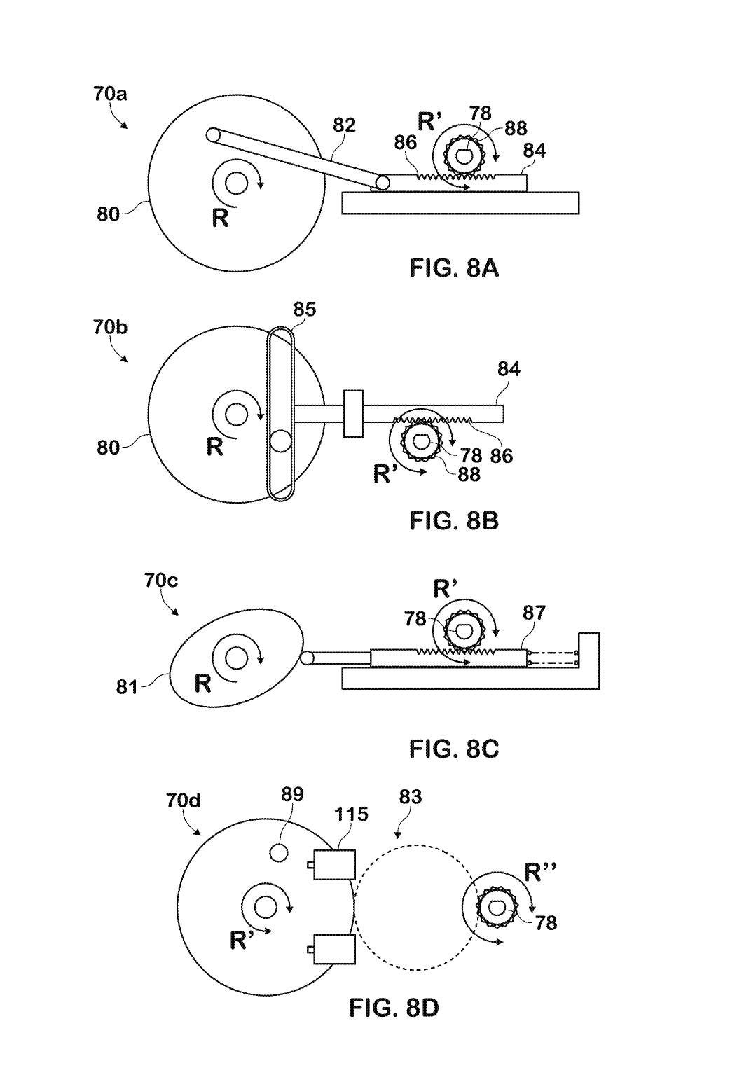

The converter 70 can use other mechanisms to convert rotation from the motor 60 into reciprocation of the feet 24a-b. In one arrangement diagrammed in FIG. 8A, the converter 70a includes a crank and slider mechanism having a crank wheel 80 and a crank arm 82. The crank wheel 80 is rotatable with a single direction rotation R in response to the motor’s drive. The crank arm 82 is pivotably connected to the crank wheel 80 and to a slider 84. The slider 84 has a rack gear 86 connected to a pinion gear 88 on the axle 78 of the articulating feet. As the crank wheel 80 rotates, the crank arm 82 pushes and pulls the slider 84 so that the rack and pinion gears 86, 88 turn the axle 78 back and forth in reciprocating rotation R’.

In another arrangement diagrammed in FIG. 8B, the converter 70b includes a scotch yoke mechanism having a crank wheel 80 and a reciprocating shaft 84. The crank wheel 80 is rotatable with a single direction rotation R in response to the motor’s drive. The wheel 80 has pin engaged in a slot 85 of the reciprocating shaft 84. As shown, the reciprocating shaft 84 can have a rack gear 86 connected to a pinion gear 88 on the axle 78 for a foot. As the crank wheel 80 rotates, the reciprocating shaft 84 reciprocates back and forth so that the rack and pinion gears 86, 88 turn the axle 78 back and forth in reciprocating rotation R’.

In yet another arrangement diagramed in FIG. 8C, the converter 70c includes a cam and follower mechanism. A cam wheel 81 is rotatable with a single direction rotation R in response to the motor’s drive. The cam wheel 81 engages a follower 87 which is biased toward the cam wheel 81. The rotating cam wheel 81 pushes the follower 87 back and forth. The follower 87 can have a rack gear connected to a pinion gear on the axle 78 for a foot. As the cam wheel 81 rotates, the follower 87 moves back and forth so that the rack and pinion gears turn the axle 78 back and forth in reciprocating rotation R’.

In other mechanisms, the converter 70 can use a Geneva wheel with linear motion. For example, a rotating wheel can drive another wheel in discrete steps, which can then drive a linear mechanism. The converter 70 can use a lead screw with a reversal mechanism. For example, the motor’s drive can rotate a lead screw that drives a nut or a slider along the lead screw. When the nut or slider reaches the end of travel, a limit switch or a mechanical stop can reverse the motor’s rotation.

FIG. 8D diagrams another mechanism for the converter 70d. Limit switches 115 are used with gears 83 to change the back-and-forth rotation R’ of the motors’ drive into back-and-forth rotation R” of the axle 78 for the foot (24a-b). One of the gears 83 can have a mechanical stop 89 that engages the limit switches 115 at ends of rotation to reverse the motor’s rotation R’ so the axle 78 back and forth in reciprocating rotation R’. The mechanisms of FIGS. 8A-8D as well as others available in the art can be used by the converter 70 to convert the rotation of the motor (60) to reciprocation of the feet (24a-b).

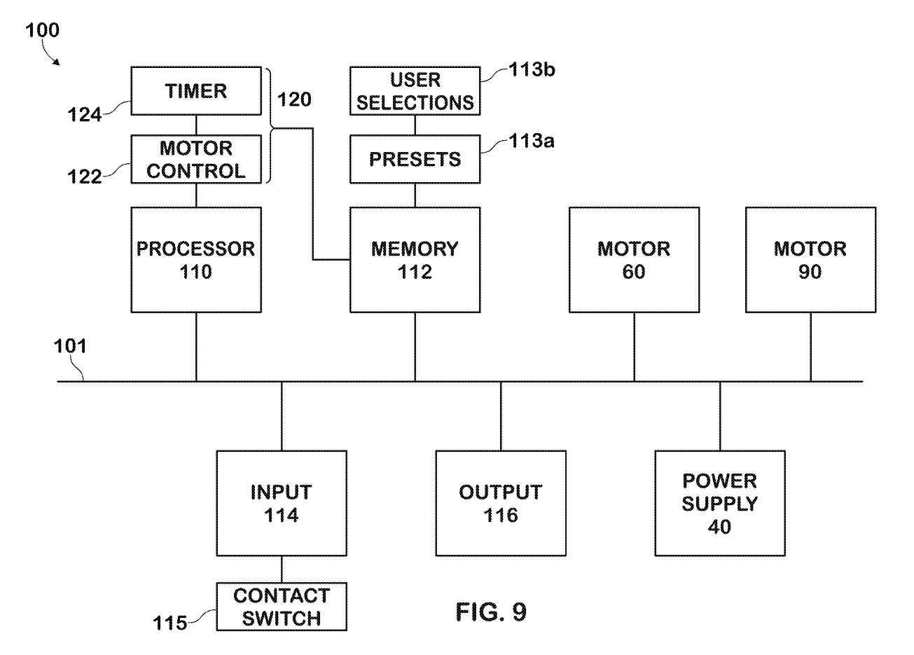

FIG. 9 schematically illustrates control circuitry 100 for the disclosed waterfowl decoy assembly 10. As already mentioned, the control circuitry 100 can be housed in the anchor (16) or in the sealed housing (25) of the decoy (20), or portions of the control circuitry 100 can be housed in both. The control circuitry 100 can include a bus 101, a processor 110, a memory 112, an input component 114, and an output component 116.

The bus 101 includes one or more components that enable wired and/or wireless communication among the components of the control circuitry 100. The bus 101 can couple together two or more components of the control circuitry 100, motor 60, and power supply 40 via operative coupling, communicative coupling, electronic coupling, and/or electric coupling. The processor 110 can include one or more of a microprocessor, a microcontroller, a field-programmable gate array, an application-specific integrated circuit, and/or another type of processing component. The processor 110 can be implemented in hardware, firmware, or a combination of hardware and software.

The memory 112 includes volatile and/or nonvolatile memory. For example, the memory 112 can include random access memory (RAM), read only memory (ROM), a flash memory, and/or another type of memory. The memory 112 stores information, instructions, and/or software 120 related to the operation of the control circuitry 100. The software 120 can include a motor control 122 to control the motor 60 and can include one or more timers 124 to time the operation of the decoy assembly (10). The input component 114 enables the control circuitry 100 to receive input, such as user input and/or sensed input. The output component 116 enables the control circuitry 100 to provide output, such as via a display, a speaker, and/or a light-emitting diode.

For example, the input component 114 can include a button, a switch, a sensor, an accelerometer, a gyroscope, and/or an actuator. Buttons and switches can be used to turn the control circuitry 100 on and off and to set user selections for operation. Sensors can detect parameters of the decoy (20) during operation. As one example, the control circuitry 100 can include limit switches 115 disposed in the sealed housing (25). The limit switches 115 can detect the limits for the rotation of the motor 60 when the direction of rotation needs to be reversed to mimic the kicking of the feet (24a-b). These and other forms of buttons, switches, and sensors can be used for the input components 114.

The control circuitry 100 can perform one or more operations or processes described herein. For example, the memory 112 can store a set of instructions (e.g., one or more instructions or code) in the software 120 for execution by the processor 110. The processor 110 can execute the set of instructions to perform one or more operations or processes described herein. In some implementations, execution of the set of instructions, by the processor 110, causes the processor 110 and/or the control circuitry 100 to perform one or more operations or processes described herein. In some implementations, hardwired circuitry can be used instead of or in combination with the instructions to perform one or more operations or processes described herein. Additionally, or alternatively, the processor 110 can be configured to perform one or more operations or processes described herein. Thus, implementations described herein are not limited to any specific combination of hardware circuitry and software.

For instance, the memory 112 can store presets 113a and user selections 113b that control the operation of the control circuitry 100, such as defining time intervals of the timer 124 used by the motor control 122 in controlling the motor 60. For instance, the timer 124 can define intervals for how long and how often the decoy (20) kicks the feet (24a-b) in the water. Additional timers 124 may define different intervals during which the motor 60 operates at different speeds to kick the feet (24a-b). An additional timer 124 may also introduce an intermediate stoppage of the motor 60. The timers 124 can have presets 113a and can be set by user selections 113b if desirable. The goal of the timers 124 is to make the kicking motion of the decoy (20) more realistic and lifelike to mimic the natural kicking motions of a duck or other waterfowl in the water.

When feeding on the water surface, ducks (especially dabbling ducks like mallards, pintails, or teal) make some distinct motions. The control circuitry 100 can control the motor 60 to kick the feet (24a-b) to mimic one or more of the distinct motions. To mimic tipping or “dabbling,” for example, while the decoy (20) is tipped forward, with the tail (22b) sticking up in the air, the control circuitry 100 can paddle feet (24a-b) on the decoy (20) in a manner as would typically be done by ducks to keep balance.

To mimic nibbling, the control circuitry 100 can control another motor 90 to produce vibration or oscillation so the decoy (20) rocks back and forth to mimic dabbling and pecking at vegetation, insects, or small critters under the water. To produce the oscillations, this motor 90 can move a weight back and forth in vertical and/or horizontal directions. Other arrangements can be used.

The foregoing description of preferred and other embodiments is not intended to limit or restrict the scope or applicability of the inventive concepts conceived of by the Applicants. It will be appreciated with the benefit of the present disclosure that features described above in accordance with any embodiment or aspect of the disclosed subject matter can be utilized, either alone or in combination, with any other described feature, in any other embodiment or aspect of the disclosed subject matter.

In exchange for disclosing the inventive concepts contained herein, the Applicants desire all patent rights afforded by the appended claims. Therefore, it is intended that the appended claims include all modifications and alterations to the full extent that they come within the scope of the following claims or the equivalents thereof.

1. An assembly to mimic a waterfowl on a water surface, the assembly comprising:
a decoy having a sealed chamber and being buoyant with at least a portion of the decoy above the water surface;
at least one articulating foot connected to the decoy;
an actuator disposed in the sealed chamber and comprising:
a motor being operable to produce a drive; and
a converter coupled between the motor and the at least one articulating foot and being configured to convert the drive of the motor into reciprocating motion of the at least one articulating foot; and
control circuitry in electrical communication with the actuator and being configured to operate the motor.

2. The assembly of claim 1, wherein the sealed chamber houses at least a portion of the control circuitry.

3. The assembly of claim 1, wherein the decoy comprises a buoyant chamber being configured to provide buoyancy to the decoy.

4. The assembly of claim 1, wherein the control circuitry comprises a power supply disposed in the sealed chamber.

5. The assembly of claim 1, further comprising an anchor useable below the water surface and having a tether connected to the decoy at the water surface.

6. The assembly of claim 5, wherein at least one of:
the anchor houses at least a portion of the control circuitry, the tether having conductors connected to the actuator; and
the anchor houses a power supply, the tether having conductors conducting power from the power supply to the actuator.

7. The assembly of claim 1, wherein the reciprocating motion of the at least one articulating foot moves at least portion of the at least one articulating foot above and below the water surface.

8. The assembly of claim 1, wherein the converter comprises a gear reducer reducing rotation of the drive from the motor.

9. The assembly of claim 1, wherein the motor produces rotation for the drive in one direction; and wherein the converter converts the rotation in the one direction to the reciprocating motion.

10. The assembly of claim 1, wherein the motor produces rotation for the drive in first and second directions opposing one another; and wherein the converter converts the rotation in the first direction into an advance of the reciprocating motion and converts the rotation in the second direction into a return of the reciprocating motion.

11. The assembly of claim 1, comprising two of the at least one articulating foot, each of the articulating feet connected at a rotatable connection to the decoy, the motor being operable to produce the drive for both of the articulating feet, the converter coupled between the motor and the articulating feet and being configured to convert the drive of the motor into the reciprocating motion of both of the articulating feet at the rotatable connections.

12. The assembly of claim 11, wherein the converter comprises crank wheels turned by the drive of the motor, each of the crank wheels being connected by a first hinge to a linkage arm, each linkage arm connected by a second hinge to a lever, each lever connected to an axle of a respective one of the rotatable connections for a respective one of the articulating feet.

13. The assembly of claim 1, wherein the at least one articulating foot is connected at a rotatable connection to the decoy; and wherein the converter comprises one of:
a crank and a slider, the crank being rotatable in response to the drive and being connected to the slider via a connecting rod, the slider being connected to the rotatable connection of the at least one articulating foot;
a scotch yoke having a wheel and a reciprocating shaft, the wheel being rotatable in response to the drive and having a pin engaged in a slot of the reciprocating shaft, the reciprocating shaft being connected the rotatable connection of the at least one articulating foot;
a cam and a follower, the cam being rotatable in response to the drive and moving the follower back and forth, the follower being connected to the rotatable connection of the at least one articulating foot;
a rack and a pinion with an oscillating link;
a rotating wheel being rotatable in response to the drive, the rotating wheel driving a Geneva wheel in discrete steps that drive a linear mechanism connected to the rotatable connection of the at least one articulating foot; and
a lead screw rotated by the drive, the lead screw being configured to move a slider connected to the rotatable connection of the at least one articulating foot, limit switches being configured to reverse rotation of the motor at end of travel of the slider.

14. The assembly of claim 1, wherein the control circuitry comprises at least one of:
a timer configured to intermittently operate the motor; and
a control configured to control a speed of the motor.

15. The assembly of claim 1, further comprising another motor disposed in the sealed chamber and being operable to produce oscillations of the decoy, the control circuitry in electrical communication with the other motor and being configured to operate the other motor.

16. An assembly for use on a water surface, the assembly comprising:
a decoy having a sealed chamber and being buoyant with at least a portion of the decoy above the water surface;
articulating feet connected at rotatable connections to the decoy;
an actuator disposed in the sealed chamber and comprising:
a motor being operable to produce a drive; and
a converter coupled between the motor and the articulating feet and being configured to convert the drive of the motor into reciprocating motion of the articulating feet at the rotatable connections;
a power supply disposed in the sealed chamber and being configured to provide power; and
control circuitry disposed in the sealed chamber and in electrical communication with the actuator and the power supply, the control circuitry being configured to operate the motor with the power from the power supply.

17. The assembly of claim 16, wherein the converter comprises crank wheels turned by the drive of the motor, each of the crank wheels being connected by a first hinge to a linkage arm, each linkage arm connected by a second hinge to a lever, each lever connected to an axle of a respective one of the rotatable connections for a respective one of the articulating feet.

18. An assembly for use on a water surface, the assembly comprising:
a decoy having a sealed chamber and being buoyant with at least a portion of the decoy above the water surface;
articulating feet connected at rotatable connections to the decoy;
an actuator disposed in the sealed chamber and comprising:
a motor being operable to produce a drive; and
a converter coupled between the motor and the articulating feet and being configured to convert the drive of the motor into reciprocating motion of the articulating feet at the rotatable connections;
an anchor useable below the water surface and connected to the decoy by a tether, the anchor having a power supply configured to provide power, the tether conducting the power from the power supply to the actuator; and
control circuitry in electrical communication with the actuator and the power supply, the control circuitry being configured to operate the motor with the power from the power supply.

19. The assembly of claim 18, wherein the converter comprises crank wheels turned by the drive of the motor, each of the crank wheels being connected by a first hinge to a linkage arm, each linkage arm connected by a second hinge to a lever, each lever connected to an axle of a respective one of the rotatable connections for a respective one of the articulating feet.

20. The assembly of claim 18, wherein the sealed chamber of the decoy houses at least a portion of the control circuitry.
